(19) United States
(12) Patent Application Publication
(10) Pub. No.: US 2020/0069676 A1
(43) Pub. Date: Mar. 5, 2020

(54) METHODS OF SAFELY TRANSITIONING A SUBJECT TO BUPRENORPHINE

(71) Applicant: BioDelivery Sciences International, Inc., Raleigh, NC (US)

(72) Inventors: Andrew Finn, Raleigh, NC (US); Todd Kirby, Malvern, PA (US); Lynn R. Webster, Salt Lake City, UT (US)

(73) Assignee: BioDelivery Sciences International, Inc., Raleigh, NC (US)

(21) Appl. No.: 16/565,743

(22) Filed: Sep. 10, 2019

Related U.S. Application Data
(63) Continuation of application No. 16/354,558, filed on Mar. 15, 2019, now abandoned.
Application No. 16/354,558 is a continuation of application No. 15/955,532, filed on Apr. 17, 2018, now abandoned.
Application No. 15/955,532 is a continuation of application No. 15/260,980, filed on Sep. 9, 2016, now abandoned.
(60) Provisional application No. 62/216,251, filed on Sep. 9, 2015.

(51) Int. Cl. A61K 31/485; A61P 25/36; A61P 25/04

(52) U.S. Cl. CPC A61K 31/485; A61K 9/006; A61P 25/04; A61P 25/36

(57) ABSTRACT

The present invention relates to methods and compositions for treating pain in a subject and safely transitioning a subject from a full μ-opioid receptor agonist to a partial μ-opioid receptor agonist.

### CROSS-RELATED APPLICATIONS

This application is a continuation of U.S. patent application Ser. No. 15/260,980, filed Sep. 9, 2016, which claims the benefit of U.S. Provisional Application Ser. No. 62/216,251, filed on Sep. 9, 2015, the entire contents of each of which are incorporated by reference herein.

### FIELD

The present invention relates to methods and compositions for treating pain in a subject and safely transitioning a subject from a full μ-opioid receptor agonist to a partial μ-opioid receptor agonist.

### BACKGROUND

Buprenorphine is a synthetic opioid which is classified as a Schedule III controlled substance in the United States. Although buprenorphine is a partial μ-opioid receptor agonist with low intrinsic activity at the molecular level, in clinical practice buprenorphine has demonstrated full analgesic efficacy, no analgesic ceiling, and an analgesic potency ranging from 30 to 115 times greater than oral morphine depending on the experimental model used and the formulation. The analgesic efficacy of buprenorphine is mediated by high affinity binding to, and a very slow rate of dissociation from μ-opioid receptors in the central nervous system. Together, these properties account for the unique profile of buprenorphine compared to other opioids including a long duration of action, a lower incidence of side effects, decreased abuse potential, and a relatively low level of physical dependence.

Buprenorphine distinguishes itself from traditional full μ-opioid receptor agonists, such as morphine and fentanyl, on respiratory depression. Unlike morphine and fentanyl which exhibit linear and dose proportional respiratory depression, buprenorphine displays a non-linear dose response curve which plateaus to a ceiling effect at intravenous doses above 2 mg. Administration of 32 mg buprenorphine produced no greater respiratory depression than 16 mg buprenorphine. When combined with clinical data which confirms that the effects of buprenorphine can be fully reversed by naloxone, buprenorphine may be considered a safe and effective analgesic with limited overdose potential.

As a partial μ-opioid receptor agonist with high affinity binding, slow receptor dissociation and low intrinsic activity at the μ-opioid receptor, administration of buprenorphine to subjects with a high percentage of μ-opioid receptors occupied by a full μ-agonist, may result in displacement of the full agonist and opioid withdrawal. Current recommendations for starting buprenorphine in subjects on ATC opioid are to taper the ATC opioid to a total daily dose of 30 mg MSE before starting buprenorphine. This introduces significant compliance issues as well as the risk of reduced pain control and end of dosing interval opioid withdrawal. There is a need in the art for methods of treating subjects, and for treating pain in subjects addicted to opioids and for treating pain. Also needed in the art are method of switching subjects from μ-opioid receptor agonist to a partial μ-opioid receptor agonist safely. The methods described herein provide such methods.

### SUMMARY

Provided herein are methods of treating pain in a subject, comprising, discontinuing a full μ-opioid receptor agonist and administering a partial μ-opioid receptor agonist.

Provided herein are methods of switching a subject from a full μ-opioid receptor agonist to a partial μ-opioid receptor agonist for the treatment of pain, comprising, discontinuing a full μ-opioid receptor agonist and administering a partial μ-opioid receptor agonist.

In one embodiment, the methods include discontinuing without tapering the dose of the full agonist.

Provided herein are methods of treating opioid-dependent subjects with chronic pain receiving around the clock opioid therapy who had precipitated opioid withdrawal following a naloxone challenge, comprising administering buprenorphine HCl buccal soluble film at approximately 50% of their prescribed MSE dose, wherein the subject does not experience withdrawal.

Provided herein are methods to determine if a subject with chronic pain on opioids receiving 80 to 220 mg oral MSE can be safely transitioned to a partial μ-opioid receptor agonist at approximately 50% of their MSE dose without inducing opioid withdrawal or loss of pain control, comprising, challenging the subject with naloxone and determining precipitation of withdrawal, wherein if the subject experienced withdrawal, the subject is a candidate for transition to buprenorphine HCl buccal film at approximately 50% of the subjects MSE dose.

Provided herein are methods to determine if a subject with chronic pain on opioids receiving 80 to 220 mg oral MSE can be safely transitioned to buprenorphine HCl buccal film at approximately 50% of their MSE dose without inducing opioid withdrawal or reversing analgesic effects, comprising, challenging the subject with naloxone and determining precipitation of withdrawal, wherein if the subject experienced withdrawal, the subject is a candidate for transition to buprenorphine HCl buccal film at approximately 50% of the subjects MSE dose.

In one embodiment, buprenorphine is administered 8-12 hours after the last dose of opioid.

In one embodiment, the opioid is one or more of an IR or an ER formulation.

In one embodiment, the subject was receiving 80 mg to 220 mg of opioid full agonist.

In one embodiment, the opioid full agonist was one or more of morphine sulfate or oxycodone HCl.

In one embodiment, the partial agonist comprises buprenorphine HCl buccal film.

In one embodiment, the buprenorphine HCl buccal film comprises 300 or 400 mcg films or any combination thereof that is calculated to be equivalent to 50 percent of their full agonist dose.

In one embodiment, the opioid is an ATC opioid.

Provided herein are methods of switching an opioid-dependent subject with chronic pain to BEMA Buprenorphine, from their stable opioid dose to approximately 50% of their stable dose compared to an estimated 50% MSE of BEMA Buprenorphine.

Provided herein are methods to evaluate the effect on the “Pain Now” NRS scores when opioid dependent subjects with chronic pain are switched from a stable opioid dose to 50% of their stable dose or to 50% MSE of BEMA Buprenorphine.

Provided herein are methods to increase the safety and tolerability of taking pain drugs when opioid-dependent subjects with chronic pain are switched from a stable opioid dose to 50% of their stable dose or 50% MSE of BEMA Buprenorphine.

Other aspects and embodiments are discussed herein infra.

### BRIEF DESCRIPTION OF THE DRAWINGS

FIG. 1. Shows a study design of the examples.

FIGS. 2A-2B. Mean (±Standard Error) of Change from Baseline of Clinical Opiate Withdrawal Scale (COWS) at Selected Time Points—Per-Protocol Population.

FIGS. 3A-3B. Mean (±Standard Error) of Change from Baseline of NRS Pain Intensity Score at Selected Time Points—Per Protocol Population.

FIG. 4. Mean (±Standard Error) of Plasma Concentrations of Buprenorphine Versus Time-Safety Population.

FIG. 5. Mean (±Standard Error) of Plasma Concentrations of Norbuprenorphine Versus Time-Safety Population.

### DETAILED DESCRIPTION

Buprenorphine is a synthetic opioid which is classified as a Schedule III controlled substance in the United States. Although buprenorphine is a partial μ-opioid receptor agonist with low intrinsic activity at the molecular level, in clinical practice buprenorphine has demonstrated full analgesic efficacy, no analgesic ceiling, and an analgesic potency ranging from 30 to 115 times greater than oral morphine depending on the experimental model used and the formulation. The analgesic efficacy of buprenorphine is mediated by high affinity binding to, and a very slow rate of dissociation from μ-opioid receptors in the central nervous system. Together, these properties account for the unique profile of buprenorphine compared to other opioids including a long duration of action, a lower incidence of side effects, decreased abuse potential, and a relatively low level of physical dependence.

Buprenorphine distinguishes itself from traditional full μ-opioid receptor agonists, such as morphine and fentanyl, on respiratory depression. Unlike morphine and fentanyl which exhibit linear and dose proportional respiratory depression, buprenorphine displays a non-linear dose response curve which plateaus to a ceiling effect at intravenous doses above 2 mg. Indeed, administration of 32 mg buprenorphine produced no greater respiratory depression than 16 mg buprenorphine. When combined with clinical data which confirms that the effects of buprenorphine can be fully reversed by naloxone, buprenorphine may be considered a safe and effective analgesic with limited overdose potential.

As a partial μ-opioid receptor agonist with high affinity binding, slow receptor dissociation and low intrinsic activity at the μ-opioid receptor, administration of buprenorphine to subjects with a high percentage of μ-opioid receptors occupied by a pure μ-agonist, may result in displacement of the pure agonist and opioid withdrawal. Current recommendations for starting buprenorphine in subjects on ATC opioid are to taper the ATC opioid to a total daily dose of 30 mg MSE before starting buprenorphine. This introduces significant compliance issues as well as the risk of reduced pain control and end of dosing interval opioid withdrawal.

Study EN3409-204 was designed to determine if subjects with chronic pain receiving 80 to 220 mg oral morphine sulfate equivalents (MSE) can be safely transitioned on to buprenorphine at approximately 50% of their MSE dose without inducing opioid withdrawal or reversing analgesic effects. The selection of 50% of a subject's MSE dose was based on the standard clinical practice for switching subjects from one opioid to another, to select a starting dose of approximately 50% dose and titrate to effect.

The safety and tolerability of buprenorphine administered as Buprenorphine HCl buccal films (formerly BEMA® Buprenorphine), in this study was measured by Clinical Opiate Withdrawal Scale (COWS) score and “Pain Now” numeric rating scale (NRS) scores. The total duration of study participation was up to approximately 8 weeks including screening visit, 2 in-clinic study visits of approximately 50 hours (2 overnights) each separated by 7 to 14 days, and a follow-up phone contact 7 to 14 days after the final dose of study drug. The study was closed for non-safety related reasons.

As recited herein, the term “BEMA” is used interchangeably with “buccal film.”

It was surprisingly found that subjects with chronic pain receiving 80 to 220 mg oral morphine sulfate equivalents (MSE) were safely transitioned on to buprenorphine at approximately 50% of their MSE dose without inducing opioid withdrawal or reversing analgesic effects. As described herein, infra, methods of the present invention, i.e., switching from administration of full μ-opioid receptor agonists to administration of buprenorphine at an estimated 50% MSE of BEMA Buprenorphine, resulted in no difference in opioid withdrawal following administration of buprenorphine at an estimated 50% MSE of BEMA buprenorphine. Thus, it was surprisingly found that patients can rotate from a full μ-opioid receptor agonist to an estimated 50% MSE of BEMA buprenorphine

Provided herein are methods to switch to BEMA Buprenorphine, as measured by the COWS scores, when opioid-dependent subjects with chronic pain are switched from their stable opioid dose to approximately 50% of their stable dose compared to an estimated 50% MSE of BEMA Buprenorphine.

Provided herein are methods to evaluate the effect on the “Pain Now” NRS scores when opioid dependent subjects with chronic pain are switched from a stable opioid dose to 50% of their stable dose or to 50% MSE of BEMA Buprenorphine.

Provided herein are methods to increase the safety and tolerability of BEMA Buprenorphine when opioid-dependent subjects with chronic pain are switched from a stable opioid dose to 50% of their stable dose or 50% MSE of BEMA Buprenorphine.

### LIST OF ABBREVIATIONS AND DEFINITIONS OF TERMS

The following abbreviations and specialist terms are used in this study protocol.

TABLE 1
Abbreviations and Specialist Terms

| Abbreviation | |
|---|---|
| or Specialist | |
| Term | Explanation |
| ATC | Around-the-clock |
| BLQ | Below the limit of quantification |
| COWS | Clinical Opiate Withdrawal Scale |
| C-SSRS | Columbia-Suicide Severity Rating Scale |
| eCRF | Electronic Case Report Form |
| GCP | Good Clinical Practice |
| IEC | Independent Ethics Committee |
| IRB | Institutional Review Board |
| MedDRA | Medical Dictionary for Regulatory Activities |
| MSE | Morphine Sulfate Equivalent |
| NRS | Numerical Rating Scale |
| PCI | Potentially clinically important |
| PI | Principal Investigator |
| PK | Pharmacokinetic |
| PP | Per Protocol |
| QT/QTcF | QT interval/corrected QT interval-Fridericia |
| | formula |
| TEAE | Treatment-emergent adverse event |

Provided herein are methods of treating pain in a subject, comprising, discontinuing a μ-opioid receptor agonist and administering a partial μ-opioid receptor agonist. In some embodiments the μ-opioid receptor agonist is a full agonist.

Provided herein are methods of switching a subject from a μ-opioid receptor agonist to a partial μ-opioid receptor agonist for the treatment of pain, comprising, discontinuing a full μ-opioid receptor agonist and administering a partial μ-opioid receptor agonist.

In one embodiment, the methods include discontinuing without tapering the dose of the agonist (not the partial agonist).

Provided herein are methods of treating opioid-dependent subjects with chronic pain receiving around the clock opioid therapy who had precipitated opioid withdrawal following a naloxone challenge, comprising administering buprenorphine HCl buccal soluble film at approximately 50% of their prescribed MSE dose, wherein the subject does not experience withdrawal.

Provided herein are methods to determine if a subject with chronic pain on opioids receiving 80 to 220 mg oral MSE can be safely transitioned to a partial μ-opioid receptor agonist at approximately 50% of their MSE dose without inducing opioid withdrawal or loss of pain control, comprising, challenging the subject with naloxone and determining precipitation of withdrawal, wherein if the subject experienced withdrawal, the subject is a candidate for transition to buprenorphine HCl buccal film at approximately 50% of the subjects MSE dose.

Provided herein are methods to determine if a subject with chronic pain on opioids receiving 80 to 220 mg oral MSE can be safely transitioned to buprenorphine HCl buccal film at approximately 50% of their MSE dose without inducing opioid withdrawal or reversing analgesic effects, comprising, challenging the subject with naloxone and determining precipitation of withdrawal, wherein if the subject experienced withdrawal, the subject is a candidate for transition to buprenorphine HCl buccal film at approximately 50% of the subjects MSE dose.

In one embodiment, buprenorphine is administered 8-12 hours after the last dose of opioid. In one embodiment, buprenorphine is administered 4-18 hours after the last dose of opioid. In one embodiment, buprenorphine is administered 10-12 hours after the last dose of opioid. In one embodiment, buprenorphine is administered 4-8 hours after the last dose of opioid. In one embodiment, buprenorphine is administered 12-18 hours after the last dose of opioid.

In one embodiment, the opioid is one or more of an Immediate Release (IR) or an Extended Release (ER) formulation.

In one embodiment, the subject was receiving 80 mg to 220 mg of opioid full agonist.

In another embodiment the subject was receiving 40 mg to 440 mg of opioid full agonist.

In one embodiment, the opioid full agonist was one or more of morphine sulfate or oxycodone HCl.

In some embodiments, the full μ-opioid receptor agonist is morphine or a pharmaceutically acceptable salt thereof, fentanyl or a pharmaceutically acceptable salt thereof, oxycodone or a pharmaceutically acceptable salt thereof, hydrocodone or a pharmaceutically acceptable salt thereof, oxymorphone or a pharmaceutically acceptable salt thereof, or hydromorphone, or a pharmaceutically acceptable salt thereof.

In some embodiments, the full μ-opioid receptor agonist is morphine or a pharmaceutically acceptable salt thereof, fentanyl or a pharmaceutically acceptable salt thereof, or oxycodone or a pharmaceutically acceptable salt thereof.

In one embodiment, the partial agonist comprises buprenorphine HCl buccal film.

In one embodiment, the buprenorphine HCl buccal film comprises 300 or 400 mcg films or any combination thereof that is calculated to be equivalent to 50 percent of their full agonist dose. In other embodiments the buprenorphine HCl buccal film comprises between 10 and 1000 mcg. In a different embodiment, the buprenorphine HCl buccal film comprises 10 mcg, 100 mcg, 200 mcg, 500, mcg or 800 mcg. In other embodiments, the buprenorphine HCl buccal film comprises between about 200 and about 1500 mcg.

In one embodiment, the opioid is an ATC opioid.

Provided herein are methods of switching an opioid-dependent subject with chronic pain to BEMA Buprenorphine, from their stable opioid dose to approximately 50% of their stable dose compared to an estimated 50% MSE of BEMA Buprenorphine. In one embodiment, the dose is between about 25 to about 75% of the stable dose.

Provided herein are methods to evaluate the effect on the “Pain Now” NRS scores when opioid dependent subjects with chronic pain are switched from a stable opioid dose to 50% of their stable dose or to 50% MSE of BEMA Buprenorphine. In one embodiment the subjects are switched from a stable opioid dose to from between about 25% to about 75% of their stable dose or between about 25% to about 75% MSE of BEMA Buprenorphine

Provided herein are methods to increase the safety and tolerability of taking pain drugs when opioid-dependent subjects with chronic pain are switched from a stable opioid dose to 50% of their stable dose or 50% MSE of BEMA Buprenorphine. Provided herein are methods to increase the safety and tolerability of taking pain drugs when opioid-dependent subjects with chronic pain are switched from a stable opioid dose to from between about 25% to about 75% of their stable dose or from between about 25% to about 75% MSE of BEMA Buprenorphine.

The buprenorphine, or buprenorphine HCl, buccal film used herein may be made by the methods disclosed, for example, in U.S. Pat. Nos. 6,159,498; 7,579,019; and 8,147,866. The buccal films may be single layer films. The buccal films may also have two or more layers. The buccal films may also comprise one or more other ingredients In some embodiments, the buprenorphine buccal film is Belbuca*, or a generic version thereof.

In buccal films containing more than one layer, the buprenorphine may be in one or more of the layers. For example in a two layer film, the buprenorphine may be in only one layer or it may be in both layers. For example, in three layer films, the buprenorphine may be in one layer or it may be in two of the layers or it may be in all three layers. See for example, U.S. Pat. No. 8,703,177, which is incorporated by reference in its entirety.

### Example 1

This was a randomized, double-blind, double-dummy, active controlled, 2-period crossover Phase 2 study in subjects receiving ATC opioid therapy and confirmed to be opioid-dependent by naloxone challenge.

Subjects entered a 7- to 14-day screening period, during which they continued to receive their ATC opioid therapy. At visit 1 (screening), subjects signed the informed consent and were assessed for protocol eligibility including naloxone challenge described here. Subjects were administered naloxone hydrochloride IV, a μ-receptor antagonist, at various doses every 5 minutes until a subject showed signs of precipitated withdrawal (ie, COWS score ≥5). Following screening, eligible subjects returned to the clinic within 7 to 14 days to be admitted for visit 2.

Qualified subjects returned to the clinic by 1800 h (visit 2, day 1) and were housed in the clinic for 2 consecutive nights (visit 2, day 2, day 3). The subjects received their prescribed evening dose of ATC opioid under supervision of clinic personnel. The site performed assessments according to the Schedule of Events. Eligible subjects were randomized to 1 of 2 treatment sequences—AB or BA with A being 2 doses of buprenorphine at 50% of their MSE dose and B being 2 doses of active ATC opioid at 50% of the subject's prescribed total daily dose. Subjects were administered (in a double-blind, double dummy manner) a first dose of study medication and were monitored in-clinic for any signs and symptoms of opioid withdrawal. Subjects were administered a second dose of the same study medication (double-blind, double-dummy) 12 hours following the first dose.

The study dose of the ATC opioid was approximately 50% of the subject's prescribed scheduled dose and the buprenorphine was approximately equal to the estimated 50% equianalgesic dose based on estimated MSE (Table 2).

TABLE 2
Group Allocation of Study MSE Doses

| | Current MSE | 50% MSE | BEMA |
|---|---|---|---|
| MSE Dose | Total Daily | Q12 h¹ | Buprenorphine² |
| Group (n) | Dose (mg) | Dose (mg) | Study Dose (μg) |
| 1 (32) | 80-160 | 20-40 | 300 |
| 2 (16) | 161-220 | 40-55 | 450 |

¹Fifty percent (50%) of the total daily dose, administered every 12 hours (Q12 h).
²Assumes buprenorphine:morphine analgesic ratio of 100:1.
³ Assumes oxycodone:morphine ratio of 2:3.

Two (2) groups of subjects stratified by their original ATC MSE dose were recruited in a 2:1 ratio.
MSE Dose Group 1: subjects requiring between 80 mg and 160 mg MSE per day of either morphine sulfate or oxycodone HCl ATC for ≥28 days.
MSE Dose Group 2: subjects requiring between 161 mg and 220 mg MSE per day of either morphine sulfate or oxycodone HCl ATC for ≥28 days.

Subjects using IR dose forms prior to the study were administered IR doses during the study, but with a 12 hour interval between the first and second dose. Subjects using ER dose forms prior to the study were administered ER doses during the study with a 12 hour interval between the first and second dose.

During the 24-hour period following the initial study dose, no analgesics were administered with the exception of Ibuprofen 400 to 600 mg PRN to treat pain, if necessary. If a subject experienced opioid withdrawal symptoms following a study dose that required treatment, a final COWS score was recorded immediately prior to administration of commercially available open-label opioid, non-opioid analgesics, or other drugs selected at the PI's discretion. If a subject required treatment for withdrawal or exhibited a COWS score of ≥13 before receiving the second dose of study medication, they were administered their prescribed ATC opioid at 12 hours (instead of receiving the second dose of study medication) and advanced to the 24 hour time point for further procedures as outlined in the Schedule of Events The subject continued to participate in any subsequent study visits as planned.

At day 3 of visit 2, subjects underwent the 24 hour assessments and were administered their currently prescribed ATC opioid in-clinic. All subjects remained in the clinic for 12 hours (or other period, at the discretion of the PI) after resuming the prescribed ATC opioid to ensure transition back to the original analgesic regimen was adequate for pain control before being discharged to continue out-of-clinic treatment for another 7 to 14 days.

Subjects continued the use of their currently prescribed ATC opioid between visits 2 and 3. Subjects returned to the clinic 7 to 14 days later to be admitted for visit 3, where subjects underwent the same procedures as during visit 2, but received the alternate treatment.

A total of 73 subjects were enrolled to this study. Thirty-nine (39) subjects, 33 in MSE Dose Group 1 and 6 in MSE Dose Group 2, were randomized.

Randomization was stratified by MSE dose group.

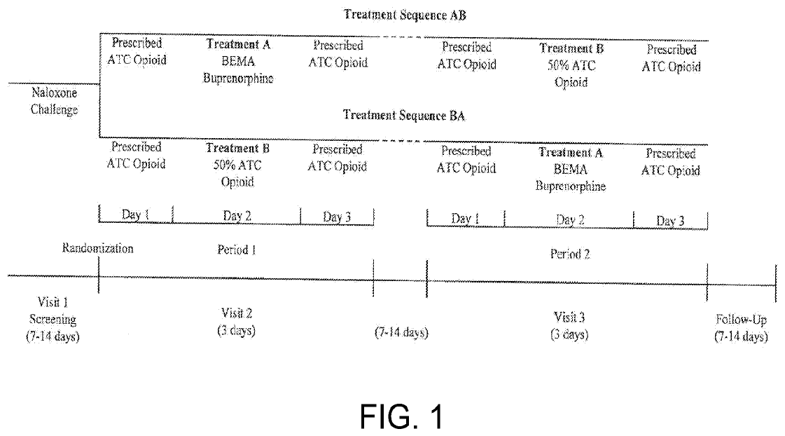

The design is depicted in FIG. 1. Subjects meeting inclusion criteria for study entry including positive precipitated withdrawal during naloxone challenge were randomized to Treatment Sequence AB or BA. Treatment A=Morning and evening administration of buprenorphine sublingual films (300 μg or 450 μg per MSE dose group) and placebo ATC capsules on Day 2; Treatment B=Morning and evening administration of 50% ATC opioid (morphine sulfate or oxycodone at 50% of prescribed total daily dose) and placebo sublingual films on Day 2.

The primary efficacy endpoint was to evaluate the tolerability of switching to buprenorphine from an ATC opioid based on COWS scores. The COWS test measures 11 opioid withdrawal symptoms in subjects physically dependent on opioids that consists of pulse rate, sweating, restlessness, pupil size, bone or joint aches, runny nose or tearing, gastrointestinal upset, tremor, yawning, anxiety or irritability, and gooseflesh skin. The secondary efficacy endpoint was to evaluate the effect on “Pain Now” using an 11-point NRS.

Safety was evaluated by monitoring adverse events (AEs), vital signs including oxygen saturation, electrocardiograms (ECGs), clinical laboratory assessments (including hematology, blood chemistry, urinalysis, drug abuse, virus serology, and pregnancy), physiological measurements, suicidality assessment (C-SSRS), opioid withdrawal symptoms (COWS), and physical examinations.

It was expected that each subject's duration of participation in the entire study would be approximately 8 weeks including a screening visit, 2 in-clinic study visits of approximately 50 hours (2 overnights) each separated by 7 to 14 days, and a follow-up phone contact 7 to 14 days after the final dose of study drug.

This interventional pain study was designed as a randomized, double-blind, double-dummy, active controlled, 2-period crossover Phase 2 safety study in opioid-dependent subjects with chronic pain receiving ATC opioid therapy who had precipitated opioid withdrawal following a naloxone challenge. This study design utilized the selection of 50% of a subject's MSE dose (who were receiving 80 to 220 mg oral MSE) based on the standard clinical practice when subjects are switched from one opioid to another (to use the approximate 50% dose as a starting dose prior to titrating up to effect) without inducing opioid withdrawal or reversing analgesic effects. Two (2) active controls used in this study were morphine and oxycodone, which are the most frequently prescribed opioids for ATC chronic pain management. The use of placebo as a control was necessary for this double-dummy study design to maintain treatment blinding and to provide reliable scientific evidence of efficacy, safety, and tolerability to ensure a reliable evaluation of the balance of benefits and risks.

For inclusion into the trial, subjects were required to fulfill all of the following criteria at visit 1, visit 2, and visit 3:
1. Written informed consent obtained prior to any study-related procedure being performed
2. Male or non-lactating female subjects 18 to 60 years of age at time of consent
3. Female subjects who were non-pregnant on the basis of screening serum pregnancy test and who were practicing abstinence or using a medically acceptable form of contraception (eg, intrauterine device, hormonal birth control, or double barrier method) or had been post-menopausal, biologically sterile, or surgically sterile (ie, hysterectomy, bilateral oophorectomy, or tubal ligation) for more than 1 year
4. Male subjects who were practicing abstinence, surgically sterile, or were using a medically acceptable form of contraception
5. Subjects with a ≥6 months history of chronic pain (including peripheral neuropathic pain) requiring ATC opioid therapy with ≥80 mg but ≤220 mg MSE per day for at least 28 days
6. Receiving one of the following opioids ATC for ≥28 days: (a) morphine sulfate; (b) oxycodone HCl
7. Display signs and symptoms of withdrawal (ie, COWS score ≥5) within 5 minutes following naloxone challenge
8. Able to understand the study procedures, complete the assessment scales, and communicate meaningfully with study personnel
9. Stable health, as determined by the principal investigator (PI), on the basis of medical history, physical examination, and screening laboratory results

Subjects were not studied if they had any of the following: a COWS score greater than 4 prior to the screening naloxone challenge; aspartate aminotransferase (AST) or alanine aminotransferase (ALT) >3 times the upper limits of normal (ULN) or serum creatinine >1.9 mg/dL at Screening, or any laboratory abnormality which, in the opinion of the Investigator, would have contraindicated study participation; use of monoamine oxidase inhibitors (MAOIs) within 14 days of screening or during the study; use of any medication, nutraceutical or herbal product with cytochrome P450 (CYP) 3A4 inhibition or induction properties within the past 30 days; documented history of alcohol and/or substance abuse (excluding nicotine and/or caffeine) within 5 years prior to screening, and/or was currently in treatment or was seeking treatment for alcohol and/or substance abuse, as assessed by the Diagnostic and Statistical Manual of Mental Disorders (DSM-IV-TR) criteria; positive alcohol breath test at screening; positive urine; or current use of α2 agonist antihypertensives (eg, clonidine), 5-HT3 antagonists (eg, ondansetron), benzodiazepines, or other medications that was anticipated to confound detection of signs and symptoms of opioid withdrawal.

Subjects were randomly assigned to 1 of 2 treatment sequences (AB, BA) to receive 1 of the following 2 double-blind, double-dummy treatments during period 1 (visit 2, day 2). The alternate double-blind, double-dummy treatment was to be administered during period 2 (visit 3, day 2).

Treatment A: Buprenorphine (active) buccal film at a total daily dose of approximately 50% the estimated equianalgesic dose of their prescribed ATC oral opioid dose and over-encapsulated placebo ATC opioid

Treatment B: Placebo buccal film and over-encapsulated active oral ATC opioid at a total daily dose of approximately 50% the prescribed ATC oral opioid dose

The study medication was provided by Sponsor as follows:
300-μg and 450-μg buprenorphine buccal films,
buprenorphine buccal films,
300-μg and 450-μg placebo buccal films,
15, 30 and 60 mg MS Contin,
15 and 30 mg Morphine Sulfate IR,
10, 15, 20, and 30 mg OxyContin, and
5, 10 and 15 mg Oxycodone IR.

Subjects were randomly assigned to identically appearing buccal films containing either buprenorphine or placebo as well as over-encapsulated tablets containing morphine sulfate (immediate-release or extended-release) or oxycodone (immediate-release or extended-release), or matching placebo in a double-blind, double-dummy manner.

Two (2) groups of subjects stratified by their ATC MSE dose (calculated at screening) were recruited in a 2:1 ratio:
MSE Dose Group 1: subjects requiring between 80 mg and 160 mg MSE per day of either morphine sulfate or oxycodone HCl ATC for ≥28 days.
MSE Dose Group 2: subjects requiring between 161 mg and 220 mg MSE per day of either morphine sulfate or oxycodone HCl ATC for ≥28 days.

As the relative analgesic potency of buprenorphine compared to oral morphine sulfate is highly variable in the literature, ranging from ×30 to ×115 depending on the experimental model and the formulation used, an analgesic potency of 100:1 was selected in this study to minimize the risk of overdosing by underestimating the potency of buprenorphine.

Buprenorphine at 300-μg and 450-μg doses were selected for this study as two doses over a 24-hour period was approximately equal 50% of the subjects MSE.

The doses used for morphine sulfate and oxycodone are as prescribed in the package inserts for the 2 drugs.

Selection and timing of dose for study drugs was based on each subject's treatment sequence. Buprenorphine HCl buccal film or matching placebo buccal film was applied by site staff to the subject's buccal mucosa in the morning and 12 hours later on day 2 at visits 2 and 3. The subjects were administered over-encapsulated oral ATC opioid or placebo tablet(s) by site staff in the morning and 12 hours later on day 2 of visits 2 and 3.

If a subject experienced opioid withdrawal symptoms at any point during the study, the subject was treated with commercially available opioid or non-opioid analgesics, or other drugs selected at the PIs discretion.

Two (2) groups of subjects stratified by their ATC MSE dose (calculated at screening) were recruited in a 2:1 ratio:
MSE Dose Group 1: subjects requiring between 80 mg and 160 mg MSE per day of either morphine sulfate or oxycodone HCl ATC for ≥28 days.
MSE Dose Group 2: subjects requiring between 161 mg and 220 mg MSE per day of either morphine sulfate or oxycodone HCl ATC for ≥28 days.

Any concomitant therapy, including the subject's currently prescribed ATC opioid use while the subject was in the study, was recorded in the source documents. From visit 2 to visit 3, subjects rated their “Pain Now” intensity according to the time points specified in the Schedule of Assessments using an 11-point NRS. The NRS anchors range from 0 to 10, where 0 represents “No pain” and 10 represents “Pain as bad as you can imagine.”

Subjects were asked: “please rate your pain now by circling the number that best describes your pain, where zero represents ‘no pain’ and 10 represents ‘pain as bad as you can imagine’.” The scores were recorded.

An AE was any unfavorable or unintended change in body structure (signs), body function (symptoms), laboratory result (eg, chemistry, ECG, X-ray, etc), or worsening of a pre-existing condition associated temporally with the use of the study medication whether or not considered related to the study medication. AEs were captured in the source documents once a subject had signed the informed consent. AEs included:
Changes in the general condition of the subject
Subjective symptoms offered by or elicited from the subject
Objective signs observed by the PI or other study personnel
All concurrent diseases that occurred after the start of the study, including any change in severity or frequency of pre-existing disease
All clinically relevant laboratory abnormalities or physical findings that occurred during the study

A treatment-emergent adverse event (TEAE) was any condition that was not present prior to treatment with study medication but appeared following treatment, was present at treatment initiation but worsened during treatment, or was present at treatment initiation but resolved and then reappeared while the individual was on treatment (regardless of the intensity of the AE when the treatment was initiated).

All AEs, including both, observed or volunteered problems, complaints, signs or symptoms must have been recorded, regardless of whether associated with the use of study medication (once the subject had taken their first dose of buprenorphine). This included AEs resulting from concurrent illness, reactions to concurrent medication use, or progression of disease states (excluding the disease under study). An AE presented at treatment initiation that worsened in intensity during treatment was re-entered with a new start date of the increased intensity. The AE was recorded in standard medical terminology when possible.

### Opioid Withdrawal

The COWS is an objective assessment of opiate withdrawal. The 11 items of the scale primarily evaluate the physical components of withdrawal and are based on questions and clinical observations. The COWS assessments were completed by the PI, physician Sub-I, or a qualified designee at the time points specified in the Schedule of Events.

The COWS scores were rated based on the sum of all 11 items as:
From 0 to 4=no withdrawal
From 5 to 12=mild
From 13 to 24=moderate
From 25 to 36=moderately severe
Greater than 36=severe withdrawal

Any signs or symptoms of opioid withdrawal which were considered to be clinically relevant were recorded in the AE section of the eCRF.

### Assessment of Pharmacokinetics

To determine buprenorphine and norbuprenorphine plasma concentrations, blood samples were drawn pre-dose and at 2 and 4 hours post dose 1 and 2 hours after dose 2.

Sample analyses for buprenorphine and its active metabolite, norbuprenorphine, were performed using a validated liquid chromatography with tandem mass spectrometry (LC/MS/MS) method.

### Efficacy Analysis

COWS consists of 11 items, each is scaled by a non-negative integer valued from 0 up to 4 or 5. The COWS total score is the summation of values of all 11 items, which ranges from 0 to 48. During each of the 2 treatment periods, COWS was measured at pre-dose (−0.5), 0.5, 1, 1.5, 2, 2.5, 3, 3.5, 4, 6, 9, 12, 12.5, 13, 13.5, 14, 16, and 24 (or end of study visit) hours post dose.

For a treatment period, a subject was defined as a responder when the subject's maximum (across all time points) COWS total score was ≥13 (or was rescued due to withdrawal symptoms) based on all available data (ie, the missing values were not imputed). Subject's response (responder: yes/no coded as I/O) was the primary endpoint of this study. The aim of this study was to estimate the odds ratio (buprenorphine:ATC opioid) in terms of the response rates.

The binary response data were analyzed using a logistic regression model with repeated measures (by subject) using Generalized Estimating Equation (GEE) methodology. The model included MSE dose group, sequence, treatment, and period as fixed effects with subject within sequence as random effect and baseline COWS total score as a covariate. The COWS data collected at −0.5 hours for a treatment were defined as the baseline value for the treatment. The estimated response rates for each treatment group, the odds ratio (buprenorphine:ATC opioid), and their corresponding 95% CIs were calculated using the model. A model with additional term for MSE dose group by treatment interaction was fitted to estimate the response rates for each treatment group, odds ratio (buprenorphine:ATC opioid) and their 95% CIs for each of the 2 MSE dose group. The interaction between MSE group and treatment was evaluated. Additionally, the estimated response rates, the response rate difference, and the corresponding 95% CIs were provided for each MSE dose group. The least square means for treatment difference and the corresponding 95% CIs for each MSE dose group were provided.

### COWS Total Scores at Appropriate Time Points

COWS total scores at each time point were summarized using descriptive statistics by treatment for each MSE dose group and overall. The mean (±SE) COWS total scores over times were plotted by treatment for each MSE dose group and overall. Scatter plots of the maximum COWS total scores for buprenorphine versus ATC opioid were also plotted; the data points were symbolized for each of the MSE dose groups.
The COWS scores were rated based on the sum of all 11 items as:
From 0 to 4=no withdrawal
From 5 to 12=mild
From 13 to 24=moderate
From 25 to 36=moderately severe
Greater than 36=severe withdrawal

COWS at each time point and maximum COWS total score were summarized using frequency and percentage based on this rating by treatment group for each MSE dose group and overall.

A linear mixed effects model with MSE dose group, sequence, period, treatment, and MSE dose group by treatment interaction effects, subject within the sequence as random effect, and baseline COWS total score as a covariate was performed for the maximum COWS total scores. The least square means for each treatment group and difference between treatment group and their corresponding 95% CIs were calculated using the model for each MSE dose group and overall.

### Numerical Rating Scale for “Pain Now”

From visit 2 to visit 3, subjects rated their “Pain Now” intensity according to the time points specified in the Schedule of Assessments using an 11-point NRS. The NRS anchors ranged from 0 to 10, where 0 represents “No pain” and 10 represents “Pain as bad as you can imagine.” The change from baseline in NRS pain score was analyzed in the same manner as that of COWS total score outlined in the previous section. The NRS pain score data collected at −0.5 hours for a treatment were defined as the baseline value for the treatment.

### Safety Analyses

Safety variables included AEs, prior and concomitant medications, vital signs, laboratory parameters, ECG parameters, physical examination, opioid withdrawal (COWS), suicidality assessment (C-SSRS), and study medication exposure.

All TEAEs that a subject reported were attributed to 1 of the 2 treatments the subject received based on the onset time of the events. A TEAE whose onset time was prior to the first dose of the second treatment period was attributed to the treatment of the first period, otherwise to the treatment of the second period. For any TEAE attributed to treatment A (or B), number of days from the first dose of A (or B) to the onset date of the event were calculated; the duration of the event was calculated as well. In case of missing or partially missing onset date, conservative philosophy dictates the determination of the attribution of an event, ie, if an attribution to a treatment for the TEAE cannot be determined, the TEAE was attributed to the treatment.

The number and percentage for subjects reporting TEAEs in each treatment group was tabulated. In these tabulations, if more than 1 TEAE was coded to the same preferred term for the same subject, the subject was counted only once for that preferred term using the most severe and most related occurrence for the summarization by severity and by relationship to the study medication, respectively. In these tabulations, the denominator for a treatment group was the total number of subjects who received the treatment.

All TEAEs were tabulated by treatment group, then by system organ class and preferred term; by treatment group, then by system organ class, preferred term, and severity; by treatment group, then by system organ class, preferred term, and relationship to study medication.

Serious adverse events (SAEs) and adverse events (AEs) leading to premature discontinuation of study medication were tabulated by treatment group, then by preferred term, and sorted by decreasing frequency for the test treatment. SAEs were displayed separately for those events occurring before the very first study medication dosing date for the entire study and all subsequent ones.

Listings were presented for subjects with any AEs, SAEs, AEs leading to discontinuation, and subjects who died (if any). A separate listing was presented for SAEs/AEs leading to screen failure resulting from naloxone challenge.

### Opioid Withdrawal

Clinical Opiate Withdrawal Scale (COWS) total scores were utilized to assess opioid withdrawal. The incidence (COWS >13), the severity for opioid withdrawal was derived based on the defined scales and summarized by treatment and MSE dose group.

### Suicidality Assessment

The following outcomes are C-SSRS categories and have binary responses (yes/no). The categories were re-ordered from the actual scale to facilitate the definitions of the composite and comparative endpoints, and to enable clarity in the presentation of the results.
Category 1—Wish to be Dead
Category 2—Non-specific Active Suicidal Thoughts
Category 3—Active Suicidal Ideation with Any Methods (Not Plan) without Intent to Act
Category 4—Active Suicidal Ideation with Some Intent to Act, without Specific Plan
Category 5—Active Suicidal Ideation with Specific Plan and Intent
Category 6—Preparatory Acts or Behavior
Category 7—Aborted Attempt
Category 8—Interrupted Attempt
Category 9—Actual Attempt (non-fatal)
Category 10—Completed Suicide

The following outcome is a numerical score derived from the C-SSRS categories.

Suicidal Ideation Score: The maximum suicidal ideation category (1-5 on the C-SSRS) present at the assessment. Assign a score of 0 if no ideation was present.

Endpoints: Composite endpoints based on the above categories are defined below.
Suicidal ideation: A “yes” answer at any time during treatment to any 1 of the 5 suicidal ideation questions (categories 1-5) on the C-SSRS.
Suicidal behavior: A “yes” answer at any time during treatment to any 1 of the 5 suicidal behavior questions (categories 6-10) on the C-SSRS.
Suicidal ideation or behavior: A “yes” answer at any time during treatment to any 1 of the 10 suicidal ideation and behavior questions (categories 1-10) on the C-SSRS.

The composite endpoints suicidality (either ideation or behavior), suicidal behavior, and suicidal ideation were summarized by treatment and MSE dose group.

### Plasma Concentrations

Blood samples for PK analysis were collected from all subjects during visits 2 and 3 predose (at −0.5 hour) and at 2, 4, and 14 hours postdose. Plasma concentrations of buprenorphine and norbuprenorphine from all subjects who received active buprenorphine were presented by subject number in a data listing. The plasma concentrations of buprenorphine and norbuprenorphine were summarized by scheduled time points and MSE dose group using N (number of subjects), mean, geometric mean, coefficient of variation (% CV), standard deviation (SD), minimum, median, and maximum.

For concentration summary statistics and plots, plasma concentrations below the limit of quantification (BLQ) were set to zero; however, BLQ concentrations between 2 non-BLQ concentrations were set to missing.

Of the 39 stratified subjects, 33 subjects were randomized in MSE Dose Group 1 and 6 subjects were randomized in MSE Dose Group 2. In MSE Dose Group 1, 31 subjects (93.9%) completed the study. Two (2) subjects, 1 each during buprenorphine treatment and ATC opioid treatment, discontinued the study due to an AE and were lost to follow-up

TABLE 3
Subject Disposition by Treatment in Randomized Subjects

| MSE Dose Group 1 (80-160 mg) | | |
|---|---|---|
| | Buprenorphine | ATC |
| | (300 μg) | Opioid |
| | n (%) | n (%) |

| | | | | |
|---|---|---|---|---|
| Randomized | 33 | (100.0) | 33 | (100.0) |
| Received study medication | 32 | (97.0) | 32 | (97.0) |
| Not exposed to randomized | 1 | (3.0)^{a} | 1 | (3.0)^{b} |
| medication | | | | |
| Completed | 31 | (93.9) | 31 | (93.9) |
| Discontinued | 1 | (3.0) | 1 | (3.0) |

| | | | |
|---|---|---|---|
| Adverse Event | 1 | (3.0)^{b} | 0 |

| Lack of Efficacy | 0 | 0 | |
|---|---|---|---|
| Protocol Violation | 0 | 0 | |
| Withdrawal by Subject | 0 | 0 | |
| Lost to Follow-up | 0 | 1 | (3.0)^{a} |

| | | |
|---|---|---|
| Other | 0 | 0 |

^{a}A Subject received ATC opioid at visit 2, but did not return for visit 3 to receive buprenorphine (300 μg).
^{b}Another Subject received buprenorphine (300 μg) at visit 2, but did not return for visit 3 to receive ATC opioid due to a serious adverse event.
^{c} A Subject received buprenorphine (450 μg) at visit 2, but did not return for visit 3 to receive ATC opioid.
Note:
Percentages are based on the number of randomized subjects.
ATC = Around the clock;
MSE = Morphine sulfate equivalent;
SAE = Serious adverse event

Thirty-nine (39) subjects were stratified and 33 subjects randomized in MSE Dose Group 1 and 6 subjects randomized in MSE Dose Group 2. The Safety Population consisted of all 39 randomized subjects. The Safety Population was used for PK analysis.

All subjects had received at least 1 prior medication before enrollment on this study. All subjects had previously received natural opium alkaloids. Other than these, the most frequent prior medications (≥15% of subjects) included benzodiazepine derivatives (46.2%), other centrally acting agents (33.3%), other analgesics and antipyretics (30.8%), benzodiazepine related drugs (23.1%), other antidepressants (23.1%), anilides (20.5%), selective serotonin reuptake inhibitors (20.5%), sympathomimetics (17.9%), propionic acid derivatives (17.9%), proton pump inhibitors (17.9%), medical gases (17.9%), and vitamin D and analogues (15.4%).

All subjects had received at least 1 concomitant medication and natural opium alkaloids while on study. Other most frequent concomitant medications (≥15% of subjects) included other centrally acting agents (33.3%), anilides (28.2%), other analgesics and antipyretics (25.6%), propionic acid derivatives (25.6%), benzodiazepine derivatives (25.6%), benzodiazepine related drugs (23.1%), other antidepressants (23.1%), selective serotonin reuptake inhibitors (20.5%), sympathomimetics (20.5%), proton pump inhibitors (17.9%) and vitamin D and analogues (15.4%).

For the PP Population, 2 subjects out of 31 subjects in Group 1 were responders (either rescued or had a COWS total score ≥13) during one or both study treatments (Table 4). Due to the very few responders (≤5), an odds ratio and the corresponding P value were not estimable. There were no responders in Group 2.

TABLE 4
Comparison of Response Rate in Maximum Clinical Opiate Withdrawal
Scale (COWS) Total Score- Per-protocol Population

| | | | Odds Ratio (95% |
|---|---|---|---|
| | Responders¹ (%) | | CI) |
| MSE Dose Group | Buprenorphine | ATC Opioid² | [BEMA:ATC] |
| Overall | 1 (2.9%) | 2 (5.7%) | Not Estimable |
| MSE Dose Group 1 | 1 (3.2%) | 2 (6.5%) | Not Estimable |
| (80-160 mg) | | | |

¹Responder is defined as a subject whose maximum (across all time points) COWS total score is >=13 or has been rescued. CIs are generated using GEE methodology (Diggle, Liang and Zeger, 1994). The model includes sequence, treatment, period as fixed effects and baseline COWS total as a covariate and subject within sequence as random effect.
²ATC Opioid: Morphine Sulfate/Oxycodone
The comparison of maximum COWS total score is presented in Table 5. The maximum COWS total score between buprenorphine and ATC opioid was similar in either MSE dose group.

TABLE 5
Comparison of Maximum Clinical Opiate
Withdrawal Scale (COWS) Total Score

| | | | | Treatment |
|---|---|---|---|---|
| | | BEMA | | Difference/ |
| | | Bupre- | ATC | (95% CI)/ |
| MSE Dose Group | Statistics | norphine | Opioid ^{[2]} | P-value [1] |
| MSE Dose Group 1 | n | 31 | 31 | −0.182 |
| (80-160 mg) | Mean | 4.6 (3.15) | 5.3 (4.42) | (−1.60, 1.24) |
| | (SD) | | | |

The change from baseline in maximum COWS total score is presented in Table 6. The change from baseline in maximum COWS total score between buprenorphine and ATC opioid was similar in either MSE dose group.

TABLE 6
Change from Baseline in Maximum Clinical Opiate
Withdrawal Scale (COWS) Total Score

| | | | | Treatment |
|---|---|---|---|---|
| | | BEMA | | Difference/ |
| | | Bupre- | ATC | (95% CI)/ |
| MSE Dose Group | Statistics | norphine | Opioid ^{[2]} | P-value [1] |
| MSE Dose Group 1 | n | 31 | 31 | −0.182 |
| (80-160 mg) | Mean | 4.0 (2.92) | 3.9 (4.11) | (−1.60, 1.24) |
| | (SD) | | | |

### COWS Total Scores at Appropriate Time Points

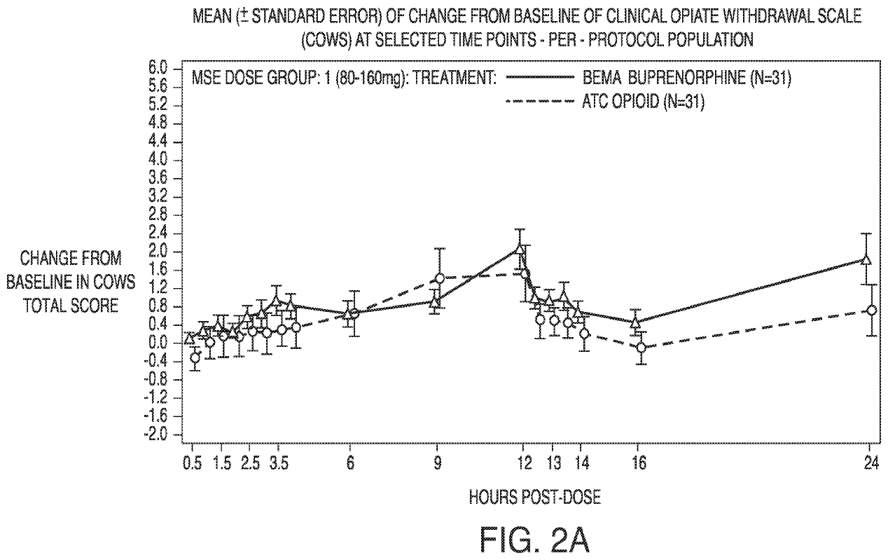

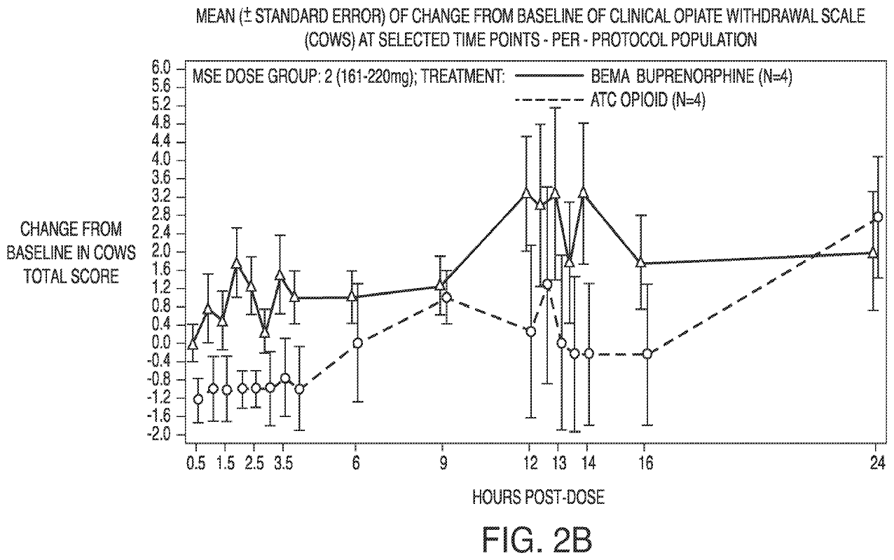

COWS total score at selected time points with treatment difference, CI, and P value is graphically depicted in FIG. 2 for the PP Population. The mean change in COWS total score from baseline during the course of 24 hours was similar for both the treatment groups with low mean changes.

COWS severity data are available for the PP Population from baseline or prior to treatment to various time points leading up to 24 hours (Table 7). No subject had a COWS total score of ≥13 up to 6 hours. Two (2) subjects (1 each at 9 hours and 12 hours during ATC opioid therapy in MSE Dose Group 1 reported a COWS score in the moderate severity range of 13 to 24. At 24 hours, no subject had a COWS total score of ≥13.

As shown in Table 7, the % of subjects with mild opioid withdrawal (COWS >5, but <13) was greater on buprenorphine than ATC opioid only at 12 and 24 hours, indicating end of dosing interval withdrawal. At all other evaluation points, either there was no difference between the study treatments in the number of subjects with mild withdrawal symptoms or there were more AT opioid subjects with mild withdrawal.

In summary, each of 3 responses occurred ≥9 hours post study dose, suggesting that they were not precipitated withdrawal, but end of dose failure. There was no difference in COWS total score following second dose in each treatment period.

Similarly, COWS total score at selected time points along with the treatment difference, CI, and P values were calculated, as were COWS severity data are available at various time points leading up to 24 hours for the Safety Population. Other than the 2 responders in the PP Population, 2 additional subjects, who were excluded from the PP Population but were a part of the Safety Population, reported a COWS total score of ≥13 (Table 7).

TABLE 7
Number (%) of Subjects by COWS Severity at Selected
Time Points- Per-protocol Population

| | | MSE Dose Group 1 (80-160 mg) | | | | | |
|---|---|---|---|---|---|---|---|
| | | Buprenorphine | | | | | |
| Time | COWS | 300 μg | | ATC Opioid | | Overall | |
| Point | Severity | N = 31 | | N = 31 | | N = 62 | |
| Baseline/ | 0-4 (No withdrawal) | 31 | (100) | 29 | (93.5) | 60 | (96.8) |
| Prior to | 5-12 (Mild) | 0 | | 2 | (6.5) | 2 | (3.2) |
| treatment | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 0.5 | 0-4 (No withdrawal) | 31 | (100) | 30 | (96.8) | 60 | (96.8) |
| (Observed) | 5-12 (Mild) | 0 | | 1 | (3.2) | 1 | (1.6) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 1 | 0-4 (No withdrawal) | 31 | (100) | 28 | (90.3) | 59 | (95.2) |
| (Observed) | 5-12 (Mild) | 0 | | 3 | (9.7) | 3 | (4.8) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 1.5 | 0-4 (No withdrawal) | 30 | (96.8) | 27 | (87.1) | 57 | (91.9) |
| (Observed) | 5-12 (Mild) | 1 | (3.2) | 4 | (12.9) | 5 | (8.1) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 2 | 0-4 (No withdrawal) | 31 | (100.0) | 27 | (87.1) | 58 | (93.5) |
| (Observed) | 5-12 (Mild) | 0 | | 4 | (12.9) | 4 | (6.5) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 2.5 | 0-4 (No withdrawal) | 30 | (96.8) | 29 | (93.5) | 59 | (95.2) |
| (Observed) | 5-12 (Mild) | 1 | (3.2) | 2 | (6.5) | 3 | (4.8) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 3 | 0-4 (No withdrawal) | 30 | (96.8) | 28 | (90.3) | 58 | (93.5) |
| (Observed) | 5-12 (Mild) | 1 | (3.2) | 3 | (9.7) | 4 | (6.5) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 3.5 | 0-4 (No withdrawal) | 28 | (90.3) | 27 | (87.1) | 55 | (88.7) |
| (Observed) | 5-12 (Mild) | 3 | (9.7) | 4 | (12.9) | 7 | (11.3) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 4 | 0-4 (No withdrawal) | 29 | (93.5) | 27 | (87.1) | 56 | (90.3) |
| (Observed) | 5-12 (Mild) | 2 | (6.5) | 4 | (12.9) | 6 | (9.7) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 6 | 0-4 (No withdrawal) | 30 | (96.8) | 28 | (90.3) | 58 | (93.5) |
| (Observed) | 5-12 (Mild) | 1 | (3.2) | 3 | (9.7) | 4 | (6.5) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 9 | 0-4 (No withdrawal) | 28 | (90.3) | 23 | (74.2) | 51 | (82.3) |
| (Observed) | 5-12 (Mild) | 3 | (9.7) | 7 | (22.6) | 10 | (16.1) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 12 | 0-4 (No withdrawal) | 26 | (83.9) | 25 | (80.6) | 51 | (82.3) |
| (Observed) | 5-12 (Mild) | 5 | (16.1) | 4 | (12.9) | 9 | (14.5) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 12.5 | 0-4 (No withdrawal) | 30 | (96.8) | 27 | (87.1) | 57 | (91.9) |
| (Observed) | 5-12 (Mild) | 1 | (3.2) | 2 | (6.5) | 3 | (4.8) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 13 | 0-4 (No withdrawal) | 30 | (96.8) | 28 | (90.3) | 58 | (93.5) |
| (Observed) | 5-12 (Mild) | 1 | (3.2) | 1 | (3.2) | 2 | (3.2) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 13.5 | 0-4 (No withdrawal) | 29 | (93.5) | 27 | (87.1) | 56 | (90.3) |
| (Observed) | 5-12 (Mild) | 2 | (6.5) | 2 | (6.5) | 4 | (6.5) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 14 | 0-4 (No withdrawal) | 31 | (100) | 28 | (90.3) | 59 | (95.2) |
| (Observed) | 5-12 (Mild) | 0 | | 1 | (3.2) | 1 | (1.6) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 16 | 0-4 (No withdrawal) | 31 | (100) | 28 | (90.3) | 59 | (95.2) |
| (Observed) | 5-12 (Mild) | 0 | | 1 | (3.2) | 1 | (1.6) |
| | 13-36 | 0 | | 0 | | 0 | |
| | (Moderate/ | | | | | | |
| | Moderately | | | | | | |
| | severe) and >36 | | | | | | |
| | (Severe withdrawal) | | | | | | |
| Hour 24 | 0-4 (No withdrawal) | 23 | (74.2) | 27 | (87.1) | 50 | (80.6) |
| (Observed) | 5-12 (Mild) | 8 | (25.8) | 4 | (12.9) | 12 | (19.4) |

| | | | |
|---|---|---|---|
| 13-36 | 0 | 0 | 0 |
| (Moderate/ | | | |
| Moderately | | | |
| severe) and >36 | | | |
| (Severe withdrawal) | | | |

### Numerical Rating Scale for Pain Now

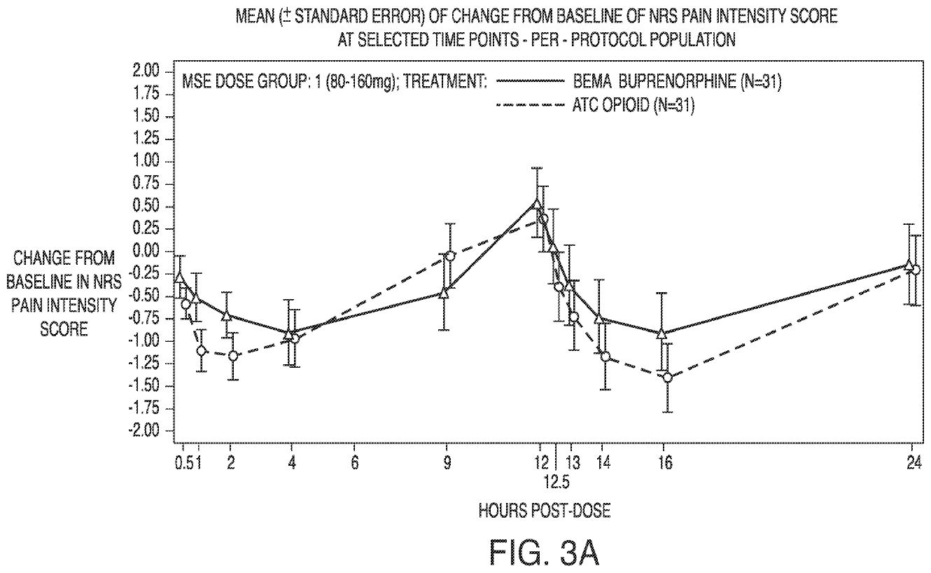

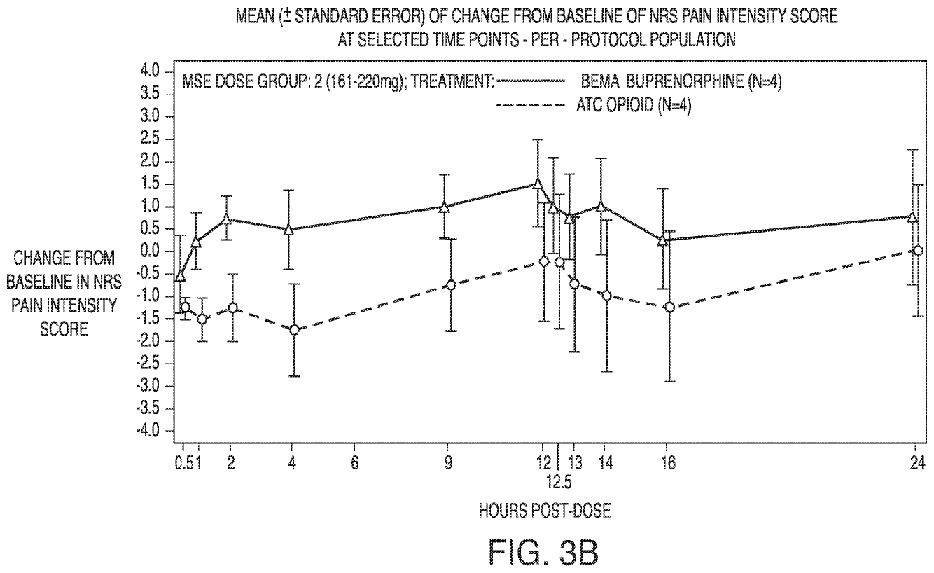

FIG. 3 shows mean change in NRS pain intensity score from baseline at selected time points in PP Population. No treatment difference is observed for either treatment group.

In MSE Dose Group 1, mean NRS values on both treatments were unchanged from baseline through 9 hours post dose. At 12 hours, there was a slight increase above baseline in both groups, that declined following dose 2 and remained low through 24 hours.

### Pharmacokinetic Analysis

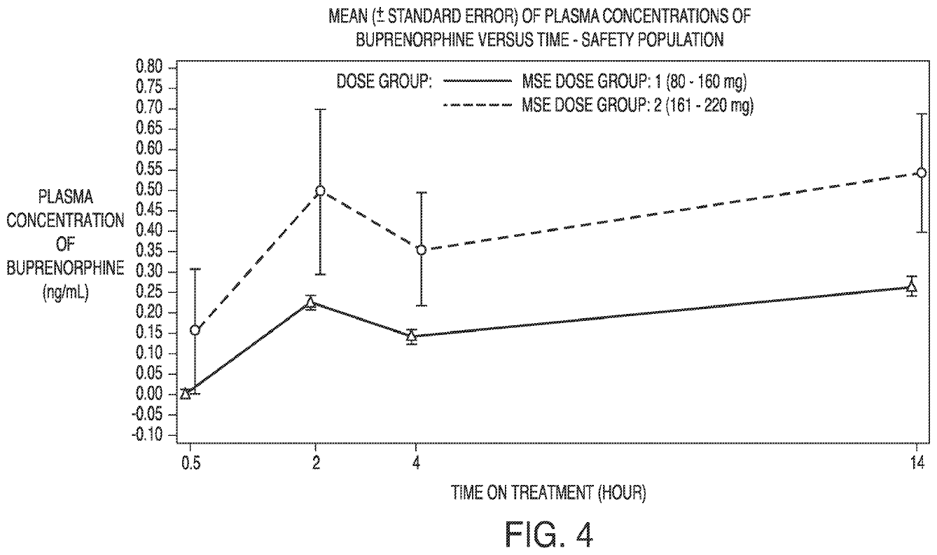

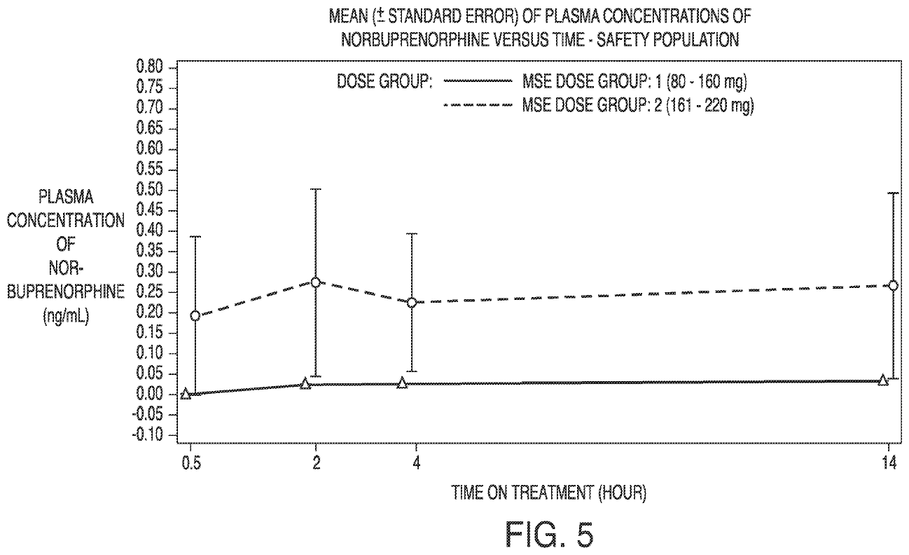

MSE Dose Group 1 (300 μg) mean plasma concentrations of buprenorphine and norbuprenorphine versus time are depicted graphically in FIG. 4 and FIG. 5, respectively. Since sampling times for this study were sparse, PK parameters, such as maximum plasma concentration (Cₘₐₓ), area under the curve (AUC), and terminal elimination half-life (t_{1/2}), were not assessed.

After administration of either buprenorphine doses, mean peak plasma concentrations of buprenorphine were observed at the first time point (2 hours) after the initial buprenorphine administration. The plasma concentrations 2 hours post dose 1 and dose 2 (14 hours) are similar indicating no accumulation from dose to dose. As would be expected buprenorphine and norbuprenorphine concentrations following the 450-μg dose were higher than those observed for the 300-μg dose.
The maximum COWS total score or change in the maximum COWS total score from baseline was similar in the two MSE dose groups.
The change from baseline in COWS total score during the 24 hour study period was similar for both the treatment groups with low mean changes.
No subject had a COWS total score of ≥13 up to 6 hours post dose in the PP Population indicating no difference between buprenorphine and ATC opioid in the risk of precipitated withdrawal.
There was no between treatment difference in pain scores over the 24-hour study periods indicating the buprenorphine doses were similar in benefit to approximately half of their ATC opioid dose. The sequence of treatment did not seem to make a difference in pain scores.
The COWS total scores indicate that subjects can switch between ATC opioid and buprenorphine in this dose range (80-220 mg MSE) without precipitating withdrawal issues. There was no evidence of a difference in precipitated opioid withdrawal following buprenorphine and ATC opioid administered 12 hours after a therapeutic dose of ATC opioid.
There was no difference in tolerability between a buprenorphine dose of 300 μg and approximately half of the prescribed ATC opioid dose administered to subjects receiving 80-160 mg ATC opioid.

The incidence of SAEs was low. Only 1 subject enrolled in MSE Dose Group 1 experienced 2 SAEs, 1 each of chest pain and dyspnea. Both of the SAEs were considered unlikely related to either study drug, buprenorphine or ATC opioid. This subject had a medical history of sarcoidosis (stage 4), chronic anterior chest pain, and dyspnea related to sarcoidosis.

TEAE occurred in 19 of 38 (50%) buprenorphine treatment periods and 13 of 37 (35%) ATC opioid treatment periods. Eleven of the TEAE on buprenorphine were considered treatment related compared to 7 of the TEAE on ATC opioid. AE leading to discontinuation occurred following exposure to buprenorphine in 2 subjects and to ATC opioid in 3 subjects.

In MSE Dose Group 1, most TEAEs were mild or moderate in intensity during treatment with buprenorphine and ATC opioid and were reported in almost similar number of subjects; mild TEAEs were reported for 13 subjects (40.6%) during treatment with buprenorphine and 10 subjects (31.3%) during ATC opioid therapy; moderate TEAEs were experienced by 4 subjects (12.5%) and 3 subjects (9.4%) during treatment with buprenorphine and ATC opioid therapy, respectively. One (1) of the 32 subjects (3.1%) in MSE Dose Group 1 experienced 2 severe TEAEs during treatment with buprenorphine, 1 each of chest pain and dyspnea. Both of these severe TEAEs were reported as SAEs. There were no severe TEAEs reported during ATC therapy.

In MSE Dose Group 1, a higher percentage of subjects had at least 1 TEAE that was considered related to the study drug according to the investigator with buprenorphine therapy (31.3%) compared to ATC opioid therapy (21.9%). The related TEAEs with buprenorphine therapy were drug withdrawal syndrome (9.4%), diarrhea (6.3%), headache (6.3%), vomiting (3.1%), arthralgia (3.1%), back pain (3.1%), nausea (3.1%), and somnolence (3.1%). The related TEAEs with ATC opioid therapy were headache (9.4%), drug withdrawal syndrome (6.3%), nausea (3.1%), and diarrhea (3.1%).

In MSE Dose Group 1, approximately a third of subjects (34.4%) reported at least 1 TEAE commonly associated with opioid use with buprenorphine therapy compared to a quarter of subjects (25.0%) with the ATC opioid therapy. TEAEs reported with buprenorphine therapy were headache (18.8%), vomiting (12.5%), nausea (9.4%), dizziness and somnolence (3.1% each). TEAEs reported with ATC opioid therapy were headache (15.6%), nausea (6.3%), and vomiting (3.1%).

A total of 9 subjects, all in MSE Dose Group 1, had TEAEs related to opiate withdrawal. All TEAEs were gastrointestinal disorders, and none of the events was serious or severe in intensity.

In subjects receiving ATC doses of 80 to 220 mg MSE, administration of 300 or 450 mcg doses of buprenorphine buccal film 8-12 hours after the last ATC dose was:
Not associated with an increased incidence of SAEs
not associated with a higher rate of discontinuations
associated with more TEAEs than the 50% ATC opioid dose
Other summary results as appropriate

These results indicate that in a controlled setting, switching from ATC opioid to buprenorphine, using standard clinical practice for selection of the initial dose, appears safe.

Buprenorphine is a partial μ-opioid receptor agonist with high affinity binding, slow receptor dissociation and low intrinsic activity, at the μ-opioid receptor. As a consequence, administration of buprenorphine to subjects with a high percentage of μ-opioid receptors occupied by a pure μ-agonist, may result in displacement of the pure agonist and opioid withdrawal. In contrast, administration of buprenorphine to subjects experiencing withdrawal, rapidly reverses the symptoms as the receptors are occupied.

Current recommendations for starting buprenorphine in subjects on ATC opioid are to taper the ATC opioid to a total daily dose of 30 mg MSE before starting buprenorphine. This introduces significant compliance issues as well as the risk of reduced pain control and end of dosing interval opioid withdrawal. This study was designed to evaluate this risk in a controlled study design with doses of buprenorphine administered 8-12 hours after the last dose of ATC opioid (IR or ER).

This study was a randomized, double-blind, double-dummy, active controlled, 2-period crossover Phase 2 study in opioid-dependent subjects with chronic pain receiving ATC opioid therapy who had precipitated opioid withdrawal following a naloxone challenge. Subjects received 2 doses of buprenorphine at approximately 50% of their MSE dose and 2 doses of active ATC opioid at 50% of their prescribed total daily dose. An aim of this study was to determine if subjects with chronic pain on ATC opioids who are receiving 80 to 220 mg oral MSE can be safely transitioned to buprenorphine HCl buccal film at approximately 50% of their MSE dose without inducing opioid withdrawal or reversing analgesic effects.

For the efficacy analysis, a subject was defined as a responder when the subject's maximum COWS total score was at least 13 (or was rescued due to withdrawal symptoms). Subject's response was the an endpoint of this study. For the PP Population, 2 subjects were responders (either rescued or had a COWS total score ≥13) during one or both study treatments. One subject had a response during buprenorphine and ATC opioid treatments and the other subject had a response during the ATC opioid treatment only. The supporting analysis of the maximum COWS scores did not show any clinically meaningful difference between the treatment groups.

The other efficacy analysis included summarization of COWS total scores at appropriate time points and NRS score for pain now. The maximum COWS total score or change in the maximum COWS total score from baseline was similar in either MSE dose group and between treatments within an MSE dose group. The change in COWS total score from baseline during the course of 24 hours was similar for both the treatment groups with low mean changes. For the PP Population, no subject had a COWS total score of ≥13 up to 6 hours post dose. The 2 responders reported a COWS score in the moderate severity range of 13 to 24 in MSE Dose Group 1. Each of 3 responses occurred ≥9 hours post study dose, suggesting that they were not precipitated withdrawal, but end of dose failure. There was no difference in COWS total score following second dose in each treatment group.

No clinically meaningful differences between treatments were observed and mean NRS scores were not much different from baseline scores indicating the effectiveness of buprenorphine therapy at 300- and 450-μg dose levels along with 50% of subjects' ATC Opioid doses. There was no between treatment difference in pain scores over the 24-hour study periods indicating the buprenorphine doses were similar in benefit to approximately half of their ATC opioid dose. The sequence of treatment did not seem to make a difference in pain scores.

The COWS total scores throughout the study duration indicate that subjects can switch between ATC opioid and buprenorphine in this dose range without precipitating withdrawal issues. There was no evidence of a difference in precipitated opioid withdrawal following buprenorphine and ATC opioid administered 12 hours after a therapeutic dose of ATC opioid.

After administration of either buprenorphine doses, mean peak plasma concentrations of buprenorphine were observed at the first time point of 2 hours after the initial buprenorphine administration. As expected, buprenorphine and norbuprenorphine concentrations following the 450 μg dose were higher than those observed for the 300-μg dose.

There were no deaths on this study. The incidence of SAEs was low. Overall, at least 1 TEAE was reported in 20 subjects (60.6%) in MSE Dose Group 1. TEAEs reported in MSE Dose Group 1 by order of frequency were associated with nervous system disorders (33.3%), gastrointestinal disorders (30.3%), general disorders and administration site conditions (18.2%), respiratory, thoracic and mediastinal disorders (6.1%), musculoskeletal and connective tissue disorders (6.1%), and investigations (3.0%).

All TEAEs, except the 2 TEAEs reported as SAEs (severe), were mild or moderate in intensity.

In MSE Dose Group 1, more subjects had at least 1 TEAE that was considered related to the study drug with buprenorphine therapy (31.3%) compared to ATC opioid therapy (21.9%). The related TEAEs with buprenorphine therapy were drug withdrawal syndrome, diarrhea, headache, vomiting, arthralgia, back pain, nausea, and somnolence. The related TEAEs with ATC opioid therapy were headache, drug withdrawal syndrome, nausea, and diarrhea.

In MSE Dose Group 1, approximately a third of subjects (34.4%) reported at least 1 TEAE commonly associated with opioid use with buprenorphine therapy compared to a quarter of subjects (25.0%) with the ATC opioid therapy. TEAEs reported with buprenorphine therapy were headache, vomiting, nausea, dizziness and somnolence. TEAEs reported with ATC opioid therapy were headache, nausea, and vomiting.

A total of 9 subjects, all in MSE Dose Group 1, had TEAEs related to opiate withdrawal. All TEAEs were gastrointestinal disorders, and none of the events was serious or severe in intensity.

No clinically meaningful trends were noted in laboratory test results, vital signs, physical examination findings, or C-SSRS.

For ECG parameters, 4 subjects in MSE Dose Group 1 had a shift from normal to abnormal during treatment. Two (2) of these changes were considered clinically significant.

The efficacy results demonstrate that opioid-dependent subjects with chronic pain receiving ATC opioid therapy who had precipitated opioid withdrawal following a naloxone challenge can be switched to buprenorphine HCl buccal soluble film at approximately 50% of their prescribed MSE dose without precipitated withdrawal.

### Example 2

Buprenorphine HCl buccal film utilizing BioErodible MucoAdhesive (BEMA®) drug delivery technology (see U.S. Pat. Nos. 6,159,498; 7,579,019; and 8,147,866, which are all incorporated by reference in their entirety). The findings showed that study participants receiving around-the-clock therapy with an opioid full agonist (morphine or oxycodone) could be switched to buprenorphine HCl buccal film, at approximately half the full agonist dose, without increasing the risk of experiencing opioid withdrawal or a loss of pain relief. This study demonstrated that study participants on a full agonist were successfully switched to buprenorphine HCl buccal film, an opioid partial agonist, at approximately 50 percent of the full agonist dose, without the need for an opioid taper and without increasing the risk of withdrawal or loss of pain control.

Switching from an Opioid Full to Partial Agonist

The Phase 2, randomized, double-blind, active controlled, 2-period crossover study included 39 chronic pain sufferers who were receiving 80 mg to 220 mg (80-160 mg, n=33; 161-220 mg, n=6) daily around-the-clock therapy of an opioid full agonist (either morphine sulfate or oxycodone HCl), and were confirmed to be opioid dependent. Approximately eight to 12 hours after the last full agonist dose, study subjects received buprenorphine HCl buccal film 300 or 400 mcg, which was calculated to be equivalent to 50 percent of their full agonist dose. The study also included an active-control group, who remained on 50 percent of their full agonist dose. The primary endpoint was the proportion of study participants that demonstrated significant withdrawal symptoms (a maximum Clinical Opiate Withdrawal Scale, or COWS, score of ≥13) or required rescue therapy because of these symptoms.

The following references are incorporated by reference, in their entirety:
1. Christoph T, Kogel B, Schiene K, Meen M, De Vry J, Friderichs E. Broad analgesic profile of buprenorphine in rodent models of acute and chronic pain. Eur J Pharmacol. 2005; 507(1-3):87-98.
2. Pergolizzi J, Aloisi A M, Dahan A, et al. Current knowledge of buprenorphine and its unique pharmacological profile. Pain Pract. 2010; 10(5):428-450.
3. Sittl R, Likar R, Nautrup B P. Equipotent doses of transdermal fentanyl and transdermal buprenorphine in patients with cancer and non-cancer pain: results of a retrospective cohort study. Clin Ther. 2005; 27(2):225-237.
4. Walsh S L, Preston K L, Stitzer M L, Cone E J, Bigelow G E. Clinical pharmacology of buprenorphine: ceiling effects at high doses. Clin Pharmacol Ther. 1994; 55(5):569-580.
5. Yassen A, Olofsen E, Romberg R, Sarton E, Danhof M, Dahan A. Mechanism-based pharmacokinetic-pharmacodynamic modeling of the antinociceptive effect of buprenorphine in healthy volunteers. Anesthesiology. 2006; 104(6):1232-1242.
6. Davis M P. Twelve reasons for considering buprenorphine as a frontline analgesic in the management of pain. J Support Oncol. 2012; 10(6):209-219.
7. Dahan A, Yassen A, Bijl H, et al. Comparison of the respiratory effects of intravenous buprenorphine and fentanyl in humans and rats. Br J Anaesth. 2005; 94(6):825-834.
8. Walsh S L, Preston K L, Bigelow G E, Sitzer M L. Acute administration of buprenorphine in humans: partial agonist and blockage effects. J Pharmacol Exp Ther. 1995; 274(1):361 372.
9. van Dorp E, Yassen A, Sarton E, et al. Naloxone reversal of buprenorphine-induced respiratory depression. Anesthesiology. 2006; 105(1):51-57.
10. Cowan A, Lewis J W, Macfarlane I R. Agonist and antagonist properties of buprenorphine, a new antinociceptive agent. Br J Pharmacol. 1977; 60(4):537-545.
11. Walsh S L, Eissenberg T. The clinical pharmacology of buprenorphine: extrapolating from the laboratory to the clinic. Drug Alcohol Depend. 2003; 70(2 Suppl): S13-27.
12. Buprenex [package insert]. Richmond, Va.: Reckitt Benckiser Pharmaceuticals, Inc.; 2010.
13. Suboxone [package insert]. Richmond, Va.: Reckitt Benckiser Pharmaceuticals, Inc.; 2012.
14. Butrans [package insert]. Stamford, Conn.: Purdue Pharma L.P.; 2012.
15. Zubsolv [package insert]. New York, N.Y.: Orexo U S, Inc.; 2013.
16. Bunavail [package insert]. Raleigh, N.C.: BioDelivery Sciences International, Inc.; 2014.
17. Budd K, Raffa R B, eds. Buprenorphine—The Unique Opioid Analgesic: Pharmacology and Clinical Application. Stuttgart, Germany: Thieme Medical Publishing; 2005.
18. M S Contin [package insert]. Stamford, Conn.: Purdue Pharma L.P.; 2012.
19. OxyContin [package insert]. Stamford, Conn.: Purdue Pharma L.P.; 2007.
20. Diggle P J, Liang K Y, Zeger S L. Analysis of Longitudinal Data. Oxford: Oxford University Press, 1994.
21. Liang K Y, Zeger S L. Longitudinal data analysis using generalized linear models. Biometrika. 1986; 73(1):13-22.

1. A method of treating pain in a patient, comprising, discontinuing a full μ-opioid receptor agonist and administering a partial μ-opioid receptor agonist at a dose that is about 50% of its prescribed MSE dose, wherein the full μ-opioid receptor agonist is discontinued without tapering prior to administration of the partial μ-opioid receptor agonist.

2-19. (canceled)
